(19) United States
(12) Patent Application Publication
(10) Pub. No.: US 2008/0262567 A1
(43) Pub. Date: Oct. 23, 2008

(54) Implanted energy source

(76) Inventors: Zohar Avrahami, Petach Tikva (IL); Yossi Gross, Moshav Mazor (IL)

Correspondence Address: HOFFMAN, WASSON & GITLER, P.C., 2461 South Clark Street, Arlington, VA, 22202, US

(21) Appl. No.: 12/153,039

(22) Filed: May 13, 2008

Related U.S. Application Data
(63) Continuation-in-part of application No. 10/891,156, filed on Jul. 15, 2004.

(51) Int. Cl. A61N 1/05

(52) U.S. Cl. 607/48

(57) ABSTRACT

Apparatus is described for use with (a) a muscle of a subject's body and (b) an implanted functional device in the subject's body. An electrode is coupled to the muscle. A stimulator causes the muscle to undergo movement by driving a current into the muscle, via the electrode. An energy assembly including a flywheel and an energy converter, stores energy associated with the movement of the muscle. The energy assembly powers the stimulator and powers the implanted functional device, based on the movement of the muscle. Other embodiments are also described.

### CROSS-REFERENCES TO RELATED APPLICATIONS

The present patent application is a continuation-in-part of U.S. patent application Ser. No. 10/891,156, filed on Jul. 15, 2004, entitled “Converting biomechanical energy to electrical/mechanical energy,” which published as US Patent Application Publication 2005/0027332, on Feb. 3, 2005, entitled “Implanted autonomic energy source,” which is incorporated herein by reference.

### FIELD OF THE INVENTION

The present invention generally relates to implanted medical apparatus. Specifically, the present invention relates to powering an implanted functional device based on the movement of a muscle.

### BACKGROUND OF THE INVENTION

Implantable functional devices that require power sources are well known in the art. For example, implantable devices are used for sensing and controlling physiological parameters, tissue stimulation, drug dispensing, external communication, and to perform functions, such as heart assistance, pumping, and neurological stimulation.

US Patent Application Publication 2005/0027332 to Avrahami et al., which is incorporated herein by reference, and of which the present application is a CIP, describes an implanted autonomic energy source which is coupled to at least one portion of a living organism that provides useful energy responsive to electrical stimulation. At least one energy converter receives the useful energy and converts it into electrical energy. Electrical stimulation means are also described. At least some of the useful energy is converted into electrical energy, energizing the electrical stimulation means, provided that the electrical energy is at least sufficient to energize the stimulation means, thus forming an electro-biological positive feedback loop that provides an autonomic useful energy source, energized by the metabolism of the living organism.

U.S. Pat. No. 3,826,265 to Giori et al., which is incorporated herein by reference, describes a medical electronic pulse generator comprising a source delivering mechanical power at a regulated rate, a motion producing means coupled to the output of the source for cyclically producing a relatively rapid mechanical motion of a duration less than that of each cycle of said pulse generator and having a consistent velocity characteristic, and a transducer means operatively associated with the motion producing means for converting the motion produced thereby to an electrical pulse. The motion producing means includes a first inertial member or element drivenly connected to the source for a portion of each cycle and freely movable during the remainder of each cycle, a second inertial member or element, and an elastic coupling means (e.g. a torsion spring) connected between the inertial members. The inertial members are stopped at predetermined times during each cycle of pulse generation, whereby the kinetic energy of one of the members after generation of a pulse is utilized to store energy in the torsion spring and to position the inertial members for the next cycle. The electrical output pulses are produced at a constant repetition frequency as determined by the mechanical power source and at a constant amplitude as determined by the consistent velocity characteristic of the motion producing means.

U.S. Pat. No. 4,661,107 to Fink, which is incorporated herein by reference, describes a pacemaker battery charging system that utilizes the pumping action of a modified heart valve to recharge the battery of the pacemaker by creating a current inductance as the valve operates, which current is supplied to the pacemaker's rechargeable battery.

U.S. Pat. No. 4,453,537 to Spitzer, which is incorporated herein by reference, describes apparatus for powering a pressure actuated body implant device. The apparatus is described as being utilized to power an artificial heart. The apparatus comprises a reservoir implantable in the body and attachable to a body muscle, a pacemaker having stimulatory electrodes for connection to the body muscle and sensor electrodes for sensing the physiological needs of the body and power output of the body muscle, and tubing for connecting the reservoir to the artificial heart. The apparatus is described as being totally implantable with the body and totally independent of any external power source.

U.S. Pat. No. 5,443,504 to Hill, which is incorporated herein by reference, describes a skeletal muscle linear pull energy converter which can harness the maximal amount of muscle power for a wide variety of implantable medical devices, including the full range of circulatory support devices. The muscle powered system is described as providing completely implantable circulatory support as an alternative to cardiac transplantation, with a quality of life relatively free from external batteries, transcutaneous energy transmission and other electromechanical hardware.

U.S. Pat. No. 5,431,694 to Snaper et al., which is incorporated herein by reference, describes a bio-operated implant system for implantation inside a human body. A piezoelectric generator in the form of a flexible sheet of poled polyvinylidene fluoride that is attached in surface-to-surface contiguity with a skeletal number, which flexes with negligible elongation of its surface, is connected in circuit with a power consuming device such as a pacemaker, to a rectifier, and to a power storage device such as a condenser or battery. The generator generates in alternating voltage, which is rectified to direct current, which is supplied to the power consuming device on demand.

The following patents, which are incorporated herein by reference, may be of interest:

U.S. Pat. No. 5,479,946 to Trumble

U.S. Pat. No. 5,810,015 to Flaherty

U.S. Pat. No. 6,640,137 to MacDonald

U.S. Pat. No. 6,470,212 to Weijand et al.

U.S. Pat. No. 5,271,395 to Wahlstrand et al.

U.S. Pat. No. 6,556,867 to Kohls

U.S. Pat. No. 6,157,861 to Faltys et al.

U.S. Pat. No. 5,109,843 to Melvin et al.

### SUMMARY OF THE INVENTION

In some embodiments of the present invention, an electrode is coupled to a muscle of a subject's body. A stimulator causes the muscle to undergo movement by driving a current into the muscle, via the electrode. An energy assembly stores energy associated with the movement of the muscle, and powers both the stimulator and an implanted functional device based on the movement of the muscle.

Typically, the energy assembly includes a flywheel and an energy converter. The flywheel stores the energy associated with the movement of the muscle by being rotated due to the movement of the muscle. The energy converter powers the stimulator and the implanted functional device based on the movement of the muscle. For example, the energy converter may convert to electrical energy, kinetic energy associated with the rotation of the flywheel. A first portion of the generated electrical energy is used to power the stimulator and a second portion of the generated electricity is used to power the implanted functional device.

There is therefore provided, in accordance with an embodiment of the invention, apparatus for use with (a) a muscle of a body of a subject and (b) an implanted functional device in the subject's body, the apparatus including:

an electrode configured to be coupled to the muscle;

a stimulator configured to cause the muscle to undergo movement by driving a current into the muscle, via the electrode; and

an energy assembly including a flywheel and an energy converter, the energy assembly configured:
to store energy associated with the movement of the muscle,
to power the stimulator based on the movement of the muscle, and
to power the implanted functional device based on the movement of the muscle.

In an embodiment:

the flywheel is configured to store kinetic energy associated with the movement of the muscle, and

the energy converter is configured:
to convert the kinetic energy to electrical energy,
to power the stimulator using a first portion of the electrical energy, and
to power the implanted functional device using a second portion of the electrical energy.

In an embodiment, the energy converter includes a magnet and a coil.

In an embodiment, the energy converter includes a piezoelectric element.

The present invention will be more fully understood from the following detailed description of embodiments thereof, taken together with the drawings, in which:

### BRIEF DESCRIPTION OF THE DRAWINGS

FIG. 1 is a block diagram of apparatus for effecting an energy loop, in accordance with an embodiment of the present invention; and

FIG. 2 is a schematic illustration of the apparatus of FIG. 1 implanted in a subject's body and effecting the energy loop of FIG. 1, in accordance with an embodiment of the present invention.

### DETAILED DESCRIPTION OF EMBODIMENTS

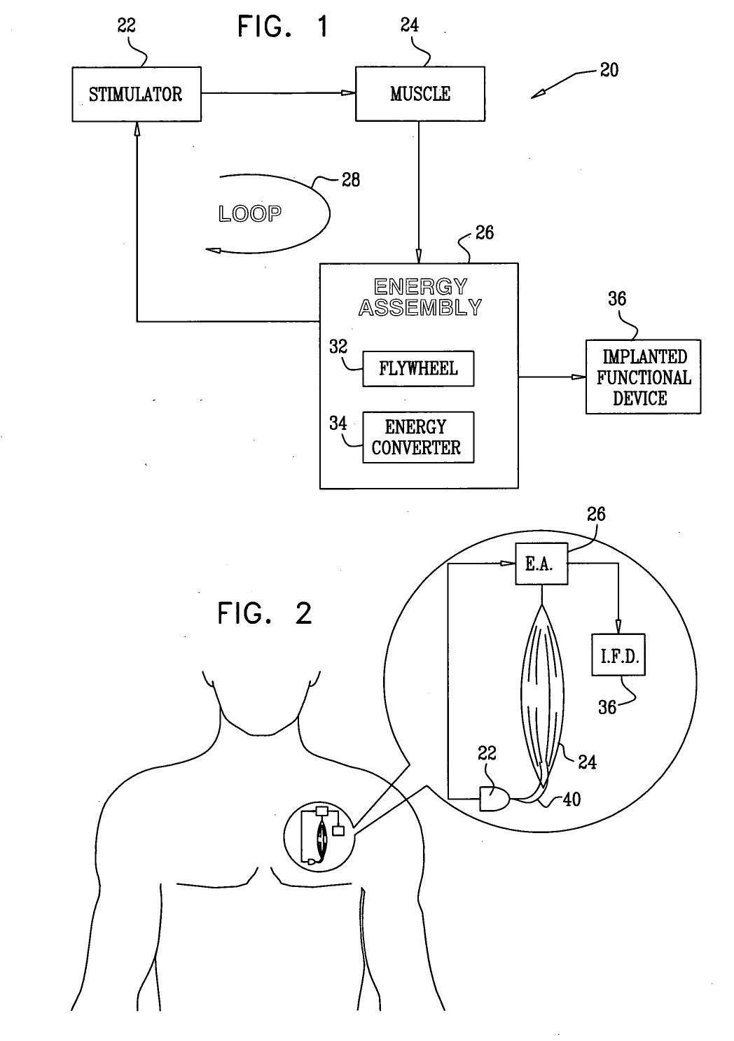

Reference is now made to FIG. 1, which is a block diagram of apparatus 20 for effecting an energy loop 28, in accordance with an embodiment of the present invention. A stimulator 22 stimulates a subject's muscle 24 causing the subject's muscle to move. An energy assembly 26 stores energy associated with the movement of the muscle, and powers both stimulator 22 and an implanted functional device 36 based on the movement of the muscle.

Typically, energy assembly 26 includes a flywheel 32 and an energy converter 34. In some embodiments, the flywheel stores the energy associated with the movement of the muscle. The energy converter powers stimulator 22 and implanted functional device 36 based on the movement of the muscle. In some embodiments, the energy assembly powers the stimulator and/or the implanted functional device using techniques described in the '322 US patent application publication to Zohar Avrahami et al., which is incorporated by reference. For example, the energy assembly may power the stimulator by moving a coil through a magnetic field of a magnet (or vice versa) in order to generate electricity, and/or by applying a force to a piezoelectric element. Typically, rotation of the flywheel due to the movement of the muscle is used to generate electricity that is used to power stimulator 22 and implanted functional device 36.

Reference is now made to FIG. 2, which is a schematic illustration of apparatus 20 implanted in a subject's body and effecting energy loop 28, in accordance with an embodiment of the present invention. Stimulator 22 stimulates muscle 24, for example by driving a current into muscle 24, via electrodes 40. Energy assembly 26 powers the stimulator 22 based on the movement of the muscle. In addition, the energy assembly powers implanted functional device 36 based on the movement of the muscle.

Typically, energy assembly 26 extracts an amount of energy from muscle 24 per loop, that exceeds the amount of energy that is used by stimulator 22 to stimulate the muscle per loop. At least a portion of the excess energy is used to power implanted functional device 36.

In some embodiments, implanted functional device 36 is configured to sense a parameter of the subject's body, control a parameter of the subject's body, stimulate tissue, dispense a drug, communicate with a device that is external to the patients body, assist cardiac function, act as a pump, and/or stimulate neurological functions of the subject.

The scope of the present invention includes all embodiments described in US Patent Application Publication 2005/0027332 to Zohar Avrahami et al., which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with (a) a muscle of a body of a subject and (b) an implanted functional device in the subject's body, the apparatus comprising:
an electrode configured to be coupled to the muscle;
a stimulator configured to cause the muscle to undergo movement by driving a current into the muscle, via the electrode; and
an energy assembly comprising a flywheel and an energy converter, the energy assembly configured:
to store energy associated with the movement of the muscle,
to power the stimulator based on the movement of the muscle, and
to power the implanted functional device based on the movement of the muscle.

2. The apparatus according to claim 1, wherein:
the flywheel is configured to store kinetic energy associated with the movement of the muscle, and
the energy converter is configured:
to convert the kinetic energy to electrical energy,
to power the stimulator using a first portion of the electrical energy, and
to power the implanted functional device using a second portion of the electrical energy.

3. The apparatus according to claim 2, wherein the energy converter comprises a magnet and a coil.

4. The apparatus according to claim 2, wherein the energy converter comprises a piezoelectric element.

5. A method for use with (a) a muscle of a body of a subject and (b) an implanted functional device in the subject's body, the method comprising:
causing the muscle to undergo movement by driving a current into the muscle;
storing energy associated with the movement of the muscle by rotating a flywheel;
continuing to power the driving of the current into the muscle based on the movement of the muscle; and
powering the implanted device based on the movement of the muscle.

6. The method according to claim 5,
wherein rotating the flywheel comprises storing kinetic energy,
wherein the method comprises converting the kinetic energy to electrical energy,
wherein continuing to power the driving of the current comprises continuing to power the driving of the current into the muscle using a first portion of the electrical energy, and
wherein powering the implanted device based on movement of the muscle comprises powering the implanted device using a second portion of the electrical energy.

7. The method according to claim 6, wherein converting the kinetic energy to electrical energy comprises electromagnetically converting the kinetic energy the electrical energy.

8. The apparatus according to claim 6, wherein converting the kinetic energy to electrical energy comprises applying a force to a piezoelectric element.
